(19) United States
(12) Patent Application Publication
(10) Pub. No.: US 2005/0249818 A1
(43) Pub. Date: Nov. 10, 2005

(54) Polycationic antimicrobial therapeutic

(76) Inventors: Samuel P. Sawan, Tyngsboro, MA (US); Tadmor Shalon, Palo Alto, CA (US); Dean Hung, Cupertino, CA (US); Norm Sokoloff, Los Altos Hills, CA (US)

Correspondence Address: PETERS VERNY JONES & SCHMITT, L.L.P., 425 SHERMAN AVENUE, SUITE 230, PALO ALTO, CA, 94306, US

(21) Appl. No.: 11/120,002

(22) Filed: May 2, 2005

Related U.S. Application Data
(60) Provisional application No. 60/567,856, filed on May 3, 2004.

(51) Int. Cl. A61K 33/38; A61K 31/155

(52) U.S. Cl. 424/618; 514/636

(57) ABSTRACT

Methods of treatment for medical indications having a microbial etiology are provided using polybiguanides, particularly water insoluble and complexed with a water insoluble antimicrobial metal material. The compositions are contacted with mucosal or dermal tissue susceptible to infection or infected in an amount sufficient to inhibit proliferation and with a spaced-apart regimen due to the persistence of the composition.

### CROSS REFERENCE TO RELATED APPLICATIONS

This application claims priority from U.S. Provisional Patent Application No. 60/567,856, filed on May 3, 2004, which is hereby incorporated by reference in its entirety.

### BACKGROUND OF THE INVENTION

1. Technical Field

The field of this invention is antimicrobial prophylaxis and therapy.

2. Background Information

There are many diseases associated with microorganisms. Bacteria and fungi are ubiquitous and have evolved along with mammalian hosts. Each of the antagonists in the battle for survival has developed mechanisms to thwart the defensive mechanisms of the other. Microorganisms have developed mechanisms of varying degrees of success to evade innate immunity, as well as the cellular and humoral immunity mechanisms of the mammalian host, resulting in infections of the host. The mammalian host substantially depends upon its immune mechanisms, but in the case of domestic animals and humans has augmented these native protective mechanisms with drugs.

Infections are defined in two basic ways: (1) related to the presence of a significant level of microbes; or (2) in relation to clinical infection as related to the presence of microbes and a host response, e.g., inflammation. In the former case, for example, infection is described as the presence of bacteria or other microorganism in sufficient quantity to damage tissue or impair healing. Clinical experience has indicated that wounds can be classified as infected when the wound tissue contains 10⁵ or greater microorganisms per gram of tissue. Clinical signs of infection may not be present, especially in the immunocompromised patient or the patient with a chronic wound. In the latter case, it is related to the presence of bacteria or other microorganisms in sufficient quantity to overwhelm the tissue defense and produce the inflammatory signs of infection, i.e. purulent exudates, odor, erythema, warmth, tenderness, edema, pain, fever and elevated white cell count. A local clinical infection is one that is confined to the wound and within a few millimeters of its margins. A systemic clinical infection is one that extends beyond the margins of the wound. Some systemic infectious complications of pressure ulcers include cellulitides, advancing cellulitides, osteomyelitis, meningitis, endocarditis, septic arthritis, bacteremia and sepsis. An inflammatory response is a localized protective response elicited by injury or destruction of tissues that serves to destroy, dilute or wall off both the injurious agent and injured tissue. Clinical signs include pain, heat, redness, swelling and loss of function. (U.S. Agency for Healthcare Policy and Research Pressure Ulcer Clinical Practice Guidelines: No. 3 & 15 (1992, 1994)).

There are many compounds that have a narrow or broad range of biocidal activity. As drugs, the compounds may act on a plurality of microorganisms, where acting on the microorganism is lethal. For the most part, these drugs are soluble and bind to or are taken up by the microorganism in order to inhibit proliferation and kill the microorganism. At the same time the compounds must have low to negligible activity against the host cells.

A known group of antimicrobials are biguanides, where the biguanides are cationic and interact with the anionic membranes of the microorganisms. The interaction can serve to compromise the membrane and allow for osmotic equilibrium and exit of essential components of the microorganism into the surrounding environment. The cationic biguanides have broad spectrum activity in view of the similarity of microorganism membrane structure. In addition, many biguanides are found not to have any significant toxicity to mammalian cells that have been tested. Numerous patents have issued where the biguanides have, for the most part, played an ancillary role in conjunction with other antimicrobials. A common biguanide that has found extensive use is chlorhexidine. Also, polyhexamethylenebiguanide has been repeatedly reported. These biguanides are for the most part water soluble and have found use as topical treatments, for example, in reducing plaque on teeth and have been impregnated in wound dressings to control bacterial populations in such dressings.

Another antimicrobial is silver, particularly as its ion. Interestingly, Silvercine is a combination of silver sulfadiazine and chlorhexidine, which has been reported to have antimicrobial activity. A nano-crystalline silver coated dressing has been reported effective against microorganisms and superior to polyhexamethylenebiguanide (“PHMB”) impregnated dressing.

In a series of patents, a non-leachable composition of polybiguanide and insoluble metal, particularly silver salts, are reported. These compounds are reported to be active against a variety of microorganisms in culture and are primarily taught as coatings, not only for devices that are introduced into the body and for containers and membranes to maintain sterility, but are also suggested to be useful for wounds. It is of interest to investigate whether these antimicrobial compositions, particularly one that is substantially insoluble in water, could serve as a therapeutic where microorganisms are involved with the etiology of the disease. These compositions would be an important adjunct to the treatment of infectious diseases that remain localized in many applications and providing long-term effectiveness against infection.

### RELEVANT LITERATURE

Wright, et al., Wounds 2003, 15, 133-42 and references cited therein describe the use of nano-crystalline silver and PHMB for use as antimicrobials in dressings. U.S. Pat. Nos. 6,180,584; 6,030632; 6,284,936; 6,126,931; 5,869,073; 5,681,468; and 5,490.938, as well as analogous foreign applications and patents; WO 01/17357; WO 00/15036; WO 99/40791; WO 98/18330; and WO 95/17152, describe the use of polybiguanides and metal antimicrobials, particularly as coatings. Charmer and Gilbert, J Appl Bacteriol 1989, 66, 253-8 describes the use of Vantocil against Providencia stuartii. Chantler, et al., Symp Soc Exp Biol 1989, 43, 325-6 report Vantocil as spermicidal. Broxton, et al., J Appl Bacteriol 1984, 57, 115-24 report Vantocil and PHMB as active against E. coli membrane. Pluss, Helv Odontol Acta 1975, 19, 61 -4 report Vantocil inhibits plaque and stains teeth. Larkin, et al., Ophthalmology 1992, 99, 185-91 reports the use of PHMB with patients having keratitis from Acanthamoeba. See also, Messick, et al., J Antimicrob Chemother 1999, 44, 297-8. In J. Clin. Periodontology 29, 392-9 a 0.12% solution is reported as a mouth rinse. Lavasept® is a combination of biguanide and polyethylene glycol and has been reported as useful in surgery as an antiseptic (Willeneger, Roth and Ochsner, 2003, Fresenius AG., D-61350 Bad Homburg).

### SUMMARY OF THE INVENTION

Polybiguanide antimicrobials, particularly in combination with insoluble metal antimicrobials, are provided for therapeutic use with microorganism associated diseases. The polybiguanide is optionally combined with an antimicrobial metal, usually as a salt. The subject antimicrobial compositions can be applied to diseased sites having a microbial component to reduce or cure the infection. The form of the formulation may be varied widely and will contain an antimicrobially effective amount of the antimicrobial composition. The subject formulations have enhanced remanence or substantivity providing for treatment over an extended period of time from a single application.

### BRIEF DESCRIPTION OF THE FIGURES

FIG. 1 depicts photographs of 1° burns and full thickness stab and staple injury, according to a general protocol as follows: Procedure: First degree burn (70 degrees C., 10 seconds) & rub (2×24), full thickness stab and staple (2×16); Innoculation: Staphlococcus, 10⁹ CFU/mL; Treatment: Neosil (1% in gel & liquid formulation; positive controls: Mupirocin & Polysporin; Negative controls: gel and liquid vehicles & no treatment; repeat treatment twice per day; Monitor: Culture by swabbing; biopsy. FIG. 1A, C: Neosil 7 days postop; FIG. 1B, D: Polysporin 7 days postop;

FIG. 2 is a bar graph comparison of CFU at different time intervals and different protocols for the study of infection of 1° burns;

FIG. 3 depicts photographs of full thickness wounds according to a general protocol as follows: Procedure: third degree burn (70 degrees C., 30 seconds) & rub (2×24), full thickness 3 mm punch bioppsy (2×24); Innoculation: Staphlococcus, 10⁹ CFU/mL;Treatment: Neosil (1% in gel & liquid formulation; positive controls: Mupirocin & Polysporin; Negative controls: gel and liquid vehicles & no treatment; repeat treatment twice per day; Monitor: Culture by swabbing; biopsy. FIG. 3A, C: Neosil 5 days postop; FIG. 3B, D: No treatment 5 days postop;

FIG. 4 is a bar graph comparison of CFU at different time intervals and different protocols for the study of infection of full thickness punch wounds;

FIG. 5 is a bar graph comparison of CFU at different time intervals and different protocols for the study of infection of partial thickness burn prophylaxis;

FIG. 6 is a bar graph comparison of CFU at different time intervals and different protocols for the study of infection of full thickness burn prophylaxis; and

FIGS. 7A and 7B report the results for survival and of CFU recovered from the mice in the comparative treatment regimens, respectively.

### DESCRIPTION OF THE SPECIFIC EMBODIMENTS

In accordance with the subject invention, stable antimicrobial compositions, particularly water-insoluble, are provided for the treatment of diseases having an etiology with a microbial component, particularly infectious diseases. The compositions comprise a polybiguanide, made generally water-insoluble by use of appropriate salts, optionally combined with a water insoluble antimicrobial metal, usually as the metal salt. The composition can be provided in various liquid or solid forms for application, using a variety of formulations for enhanced activity.

The indications involve areas of microbial invasion or infection, frequently with dermal lesions associated with sub-stratum corneum regions or mucosal regions. The subject compositions will usually be administered by techniques that do not require invasive methods for effective treatment. While the subject compositions find general application with mammalian hosts for reducing the level of microbial presence or invasion, areas of particular interest are associated with breaks in the skin barrier, e.g., open wounds, the mouth, vagina, and GI tract. Indications of interest include acne, impetigo, thrush, oral mucositis, periodontal diseases, burns, wounds, yeast infections, other fungal infections, such as vaginal infections of Candida, Gardnerella, and Trichomonas, as well as Chlamydia infections, and VRE infected GI tract. The subject compositions may also be used as surgical irrigants. The particular composition employed will depend upon the nature of the indication, the manner of application, the desired outcome, the potential for side effects, etc.

The subject compositions are polycationic polymers, particularly polybiguanide polycations, whose water solubility may be substantially reduced by selection of the appropriate anions, or complexing with a substantially water-insoluble metal or metal ion, usually metal salt, to provide complexed polybiguanides. The weight percent of the metal component of the active composition will generally be in the range of about 0 to 30%, usually at least about 0.1%, more usually in the range of about 0.5 to 20%, preferably in the range of about 1 to 15%. The weight ratio of the polybiguanide to metal, when the metal is present, will generally be in the range of about 3-1000:1, more usually in the range of about 3-200:1.

The polybiguanides have at least 2, usually at least 4, and may have 100 or more biguanides in the chain, particularly at least 4, more particularly at least 5, and not more than about 200 usually not more than about 100. The individual biguanide units will be joined by linkers of from about 2 to 12, usually 2 to 8 atoms, which may be carbon or heteroatoms, e.g., N, O, S and P, usually carbon atoms. While the linkers may be aliphatic, alicyclic, aromatic or heterocyclic, desirably they will be aliphatic, particularly a divalent alkylene. The linkers may be aliphatically saturated or unsaturated, usually saturated. A polybiguanide composition of particular interest is a polyhexamethylene biguanide available from Arch, as Cosmocil®, as available or fractionated to obtain a different average molecular weight.

The cytotoxicity and antimicrobial activity may vary with variation in the average molecular weight and the molecular weight profile. For some indications, reducing the antimicrobial activity of the polybiguanide, particularly when complexed with an antimicrobial metal or metal ion, may be desirable. In most instances, cytotoxicity of the healthy host cells will be undesirable. It is believed that antimicrobial activity and the cytotoxicity of the polybiguanide will diminish with increasing molecular weight.

The subject compositions may be obtained by fractionating commercially available mixtures of polybiguanides that may include significant amounts of biguanide. For the most part, the subject compositions will have less than about 10 weight %, usually less than about 5 weight %, of the biguanide, and may be substantially free of the biguanide. Fractions of interest include up to 1.5 kamu, 1.5 to 3 kamu, 3-5 kamu, 5-10 kamu, and greater than 10 kamu (1 kamu is equal to 1 kdal). Depending upon the application, the polybiguanide composition may be a combination of two or more of the indicated fractions that are contiguous or non-contiguous, so that the molecular weight profile may be continuous or discontinuous. Desirably suitable pharmaceutical compositions will have as the active ingredients polybiguanides of which at least 90 weight %, more usually, at least 95 weight %, have a molecular weight in the range of 1.5 kamu to 20 kamu, usually in the range of 1.5 kamu to 10 kamu.

Various conventional fractionation methods may be used, conveniently ultrafiltration with membranes having the appropriate cut-offs, ion exchange columns, liquid chromatography, fractional precipitation etc. The particular method employed will be one of convenience based on the desired fraction(s), the characteristics of the polybiguanide, and the like.

The anion for the polybiguanide will be a physiologically compatible anion, organic or inorganic. The anion may be mono- or polyvalent, hydrophilic or hydrophobic. Conveniently, the anion may reduce the water solubility of the polybiguanide to further inhibit solubilization of the subject composition. Convenient anions include halides, e.g., chloride and iodide, acetate, organic carboxylic acids, substituted or unsubstituted, e.g., gluconate, glycolate, glycinate, dodecylsulfonate, succinate, maleate, laurate, stearate, oleate, etc., or combinations thereof, where the anions will be selected to reduce or enhance the solubility of the polybiguanide-metal salt complex in one or more solvents. In various applications, one anion may be chosen over another for purposes of formulation, ease of preparation, physiological activity in the environment employed, and the like.

The metallic material can be a metal, e.g., metal particles or metal nanoparticles, metal oxide, metal salt, metal complex, metal alloy or mixture thereof, preferably a metal salt, that is capable of being transferred to a microbe on contact, but the complex does not dissolve to any significant degree, e.g., a biocidal degree, into the surrounding medium. Metallic materials which are bactericidal and are substantially water-insoluble are employed. The metallic material should be bactericidal to at least one microbe of interest and preferably will have a broad range of activity, e.g., bacterial, fungi, and protista. Examples of such metals include, e.g., silver, zinc, cadmium, lead, mercury, antimony, gold, aluminum, copper, platinum and palladium, their oxides, salts, complexes and alloys, and mixtures of these. The appropriate metallic material is chosen based upon the microbial activity in the presence of the polybiguanide. The preferred metallic materials are water insoluble silver salts that are physiologically compatible, e.g., silver iodide, phosphate, borate, bromide, etc.

The subject compositions can be prepared in a variety of ways. Where the subject composition is formulated on a surface, e.g. small particles, the particles may be coated with the metal, followed by the addition of the polybiguanide. Alternatively the metal may be reacted with an oxidant to form the salt. For example, silver may be reacted with halogen, e.g., chlorine, bromine, or iodine, and in the former cases, the resulting silver halide reacted with an iodide salt to form the silver halide. The polybiguanide may then be added in an appropriate solvent, whereby the polybiguanide will complex with the silver. In another protocol, a soluble metal salt may be combined with the polybiguanide in a suitable solvent and a non-solvent added to precipitate the complex. By adding an anion resulting in the formation of an insoluble salt, the resulting precipitate is then isolated as a water-insoluble complex. Alternatively, the polybiguanide and metal salt may be dissolved in an appropriate solvent and by evaporation, cooling, or other condition that results in separation of the combination of polybiguanide and metal salt, the product isolated.

A further alternative is for the polybiguanide and metal salt to be dissolved in water using appropriate solubilization aids. For example, the use of potassium or sodium iodide with silver iodide creates complexes that are water soluble and become water insoluble upon the evaporation of water. Further the use of coordination compounds such as PVP (polyvinylpyrrolidone), NMP or other pyrrolidones will assist in the solubilization of the metal salt. Polybiguanides may themselves be water soluble in a particular formulation and become water insoluble by combination of appropriate anions and or metal salts upon drying of the formulation.

For the metal, one may add a reductant to the salt resulting in the reduction of the metal cation to the metal. For the oxide, by adding base to an aqueous solution of the salt, the insoluble oxide forms and precipitates. In some instances, one may combine the dry compounds in the presence of a small amount of a weak solvent in an appropriate mechanical mixer and comminute the mixture to provide a homogeneous mixture and any remaining solvent removed.

Various solvents may be used, particularly organic solvents, such as alcohols, e.g., ethanol, propanol, etc., dimethylformamide, dimethylsulfoxide, N-methyl pyrrolidone, etc. Those solvents that are not physiologically acceptable at the concentration employed may be removed by evaporation. In addition, a small amount of a surfactant may be included in the solution, generally at a concentration in the range of about 0.01 to 0.5M. Various physiologically acceptable surfactants can be used, such as sodium dodecyl sulfate, sodium oleate, sodium laurate, etc., where the surfactant anion may become a component in the subject composition.

The subject compositions may be prepared in a variety of formulations, using the subject compositions by themselves or in conjunction with other therapeutic ingredients, depending upon the nature of the indication. Formulations may include gels, lotions, particles, slow release tablets, capsules, gums, powders, sprays, creams, foams, lozenges, lotions, gels, pastes, waxes, oils, ointments, soaps, etc. Particles and powders will generally be in the range of 1 micron to about 500μ, more usually not more than about 200μ. Each of the formulations will depend, for the most part, on conventional ingredients. Carriers useful in the present invention include liquids, gels, lotions, creams, ointments or foams. Liquids useful as the liquid carrier for the antimicrobial materials in the present invention include any polar liquid, including water, alcohols such as ethanol or propanol, polar aprotic solvents such as N,N-dimethyl formamide (DMF), dimethyl sulfoxide (DMSO) or N-methyl-2-pyrrolidone (NMP), and mixtures thereof. The currently preferred liquid carrier comprises a mixture of ethanol and water that may also include a solubilizing aid such as PVP or NMP. The liquid carrier in the present invention can itself be an antimicrobial disinfectant capable of causing immediate disinfection upon application of the formulation on a bacterially contaminated surface, including specially denatured alcohol (SD-alcohol) which is typically comprised of 95% ethyl alcohol denatured with 5% isopropanol, or pure isopropanol or other acceptable denaturant.

Formulations suitable for oral administration may be presented in discrete units, such as capsules, cachets, lozenges, or tablets, each containing a predetermined amount of the active compound; as a powder or granules; as a dispersion in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable apparatus, the compound in a free-flowing form, such as a powder or granules optionally mixed with a binder, lubricant, inert diluent, and/or surface active/dispersing agent(s). Molded tablets may be made by molding, in a suitable apparatus, the powdered compound moistened with an inert liquid binder.

Formulations suitable for buccal or sub-lingual administration include lozenges comprising the active compound in a flavored base, usually sucrose, and acacia or tragacanth; and pastilles comprising the compound in an inert base such as gelatin and glycerin or sucrose and acacia.

When parenteral administration is of interest, formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, or intradermal injection. Such preparations may conveniently be prepared by admixing the compound with water or a glycine buffer and rendering the resulting solution sterile and isotonic with the blood.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for transdermal administration may be presented as discrete patches adapted to remain in intimate contact with the epidermis of the recipient for a prolonged period of time. Formulations suitable for transdermal administration may be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water. Concentrations that have found application for transdermal methods have generally employed from 0.1 to 0.2M active ingredient.

Topical formulations suitable for topical application to the skin may be used in appropriate situations where the active ingredient can reach the microbial infection, and may take the form of an ointment, cream, lotion, paste, gel, spray, aerosol, lotion, shampoo, foam, cream, gel, ointment, salve, milk, stick, spray, balm, emulsion, powder, solid or liquid soap, or oil, particularly in conjunction with wounds and lesions where the area surrounding the wound or lesion is retained free of microbial invasion. Such topical formulations comprise the active compound and an acceptable carrier or medium. The acceptable carrier may comprise water or a mixture of water and at least one organic solvent which is physiologically acceptable for the purpose of a topical application. Among these solvents, exemplary are acetone, C₁-C₄ lower alcohols such as ethanol and isopropyl alcohol, alkylene glycols such as ethylene glycol and propylene glycol, ethylene glycol monomethyl, monoethyl or monobutyl ethers, the monoethyl ethers of propylene glycol and of dipropylene glycol, the C₁-C₄ alkyl esters of short-chain acids and polytetrahydrofuran ethers. When these are indeed present, such solvents preferably constitute from 1% to 80% by weight of the total weight of the formulation.

Depending on the intended application of the subject formulations, one skilled in the art can easily select the particular compounds and excipients that are necessary and characteristically employed to prepare those formulations. Among these excipients or additives, especially representative are preservatives, stabilizing agents, pH regulators, osmotic pressure modifiers, emulsifying agents, sunscreen agents, antioxidants, fragrances, colorants, anionic, cationic, nonionic, amphoteric or zwitterionic surface-active agents or mixtures thereof, viscosity modifiers, polymers, and the like.

A topical formulation of the present invention, in addition to the active compound or the pharmaceutically acceptable salt thereof and the acceptable medium or carrier, may also include an agent which enhances penetration of an active ingredient through the skin. Exemplary agents which increase skin penetration are disclosed in the following U.S. patents all of which are incorporated herein by reference: U.S. Pat. No. 4,537,776 (a binary combination of N-(hydroxyethyl)pyrrolidone and a cell-envelope disordering compound); U.S. Pat. No. 4,130,667 (using a sugar ester in combination with a sulfoxide or phosphine oxide); and U.S. Pat. No. 3,952,099 (using sucrose monooleate, decyl methyl sulfoxide, and alcohol). See also Manou et al., Acta Horticulture 344, 361-69 (1993).

Other exemplary materials that increase skin penetration are surfactants or wetting agents which include the following: polyoxyethylene sorbitan monooleoate (Polysorbate 80); sorbitan monooleate (Span 80); p-isooctyl polyoxyethylene-phenol polymer (Triton WR-1330); polyoxyethylene sorbitan trioleate (Tween 85); dioctyl sodium sulphosuccinate; and sodium sarcosinate (Sarcosyl NL-97); and other pharmaceutically acceptable surfactants.

The pharmaceutically acceptable carrier may be thickened using thickening agents typically employed in pharmaceuticals. Among these thickening agents, particularly exemplary are cellulose and derivatives thereof such as cellulose ethers, heterobiopolysaccharides such as xanthan gum, scleroglucans, and polyacrylic acids which either may or may not be cross-linked. The thickening agents are preferably present in proportions ranging from approximately 0.1% to 10% by weight relative to the total weight of the composition. The thickening agent or viscosity enhancing agent will be selected in accordance with the nature of the formulation, for example, cream, gel, viscid liquid, etc.

The dose of the compound administered to the subject in need of treatment is that amount effective to prevent the onset or occurrence of a disorder caused by microbial infection, or to treat the disorder caused by the microbial infection from which the subject suffers. By “effective amount,” “therapeutic amount,” or “effective dose,” is meant that amount sufficient to elicit the desired pharmacological effects, thus resulting in effective prevention or treatment of the disorder.

The protocol employed for the treatment will vary widely depending upon the nature of the indication, the formulation and the manner of administration. In many cases, one will not need to administer the subject compositions more frequently than about once every 4 hours and as appropriate may decrease the application to once every 8 hours, frequently not more than once every 12 hours, more frequently not more than once every day, or even less. The method of application will ordinarily be conventional for the indication being treated and the subject composition will be formulated accordingly.

Preferably, the purity of the active compounds of the present invention is greater than about 50% pure, usually greater than about 80% pure, often greater than about 90% pure, and more often greater than about 95%, 98%, or even 99% pure, with active compounds approaching 100% purity being used most often.

The effective concentration or dosage of any specific compound, the use of which is in the scope of present invention, will vary somewhat from compound to compound, patient to patient, and will depend upon the condition of the patient and the route of delivery. As a general proposition, the dosage of an active compound of the present invention at which therapeutic efficacy will be achieved may be low as about 0.10 mg/kg, but is often greater than 1 or 10 mg/kg, and typically greater than about 20 mg/kg. The dosage of the active compound may be less than about 1 g/kg, but is typically less than about 100 mg/kg, usually less than 75 mg/kg and frequently less than 50 mg/kg. Still higher dosages may potentially being employed for oral, topical, and/or aerosol administration. Toxicity concerns at the higher level may restrict intravenous dosages to a lower level such as up to about 10 mg/kg, all weights being calculated based upon the weight of the active base, including the cases where a salt is employed. Typically a dosage from about 1 mg/kg to about 50 mg/kg will be employed for intravenous or intramuscular administration. A dosage from about 1 mg/kg to about 50 mg/kg may be employed for oral administration. For topical administration, suitable concentrations of the active compound may be from 0.1 g/ml to about 500 mg/ml.

The amount of the subject compositions in the formulations will vary widely depending upon the nature of the formulation, the nature of the indication, the manner of administration, the frequency of administration, the absence or presence of other ingredients.

The active compounds of the present invention have antimicrobial (e.g., antibacterial and antifungal) activity in association with skin lesions. These compounds are useful for the treatment of conditions including, but not limited to, acne vulgaris, preadolescent acne, rosacea, premenstrual acne, acne venenata, acne cosmetica, pomade acne, acne detergicans, acne cosmetica, acne excorie, gram negative acne, steroid acne, acne conglobata, or nodulocystic acne. The present invention can also be used for topically treating certain types of dermatitis, e.g., perioral dermatitis, seborrheic dermatitis, gram negative folliculitis, sebaceous gland dysfunction, hidradenitis suppurativa, pseudofolliculitis barbae, folliculitis and dermatophyte infections (e.g., such as ringworm, athletes foot, and jock itch). The compounds are also useful in methods of preventing or ameliorating undesirable body odor.

In these applications, adjunct ingredients include, but are not limited to, not only retinoids, topical antibiotics, and benzoyl peroxide conventionally used in acne treatments, but also methyl-/ethyl-aminoalcohols, a-hydroxy acids, tyrosine tocotrienols, and fatty acid esters of ascorbic acid. Retinoids useful as adjunct ingredients include commercially available adapalene, tazarotene and/or tretinoin. See, WO 02/080932. Adapalene, for example, is currently sold as a gel or solution marketed as DifferinO. Tretinoin can be obtained as a cream, gel or encapsulated microsphere marketed as AvitaO, RenovaO, or Retin-AO. Tazarotene is marketed as a TazoracO gel. The amount of these adjunctive ingredients may be as high as their normal level of treatment, generally less than about 0.5 the normal amount and may be as little as 0.1% of the normal amount.

The polybiguanides are commercially available and find use independently or in conjunction with a metal antimicrobial. The polybiguanides can be prepared, for example, by combining a diamine with a 1,6-di(N³-cyano-N¹-guanidino)hexane prepared in accordance with Example 1 of U.S. Pat. No. 4,537,746. The resulting polybiguanide can be purified to the different chain lengths to provide the polymer of interest. The polybiguanide is water soluble and by adding an excess of a salt to the polybiguanide, particularly where the cation reacts with the anion of a polybiguanide salt or by adding an acid to the neutralized polybiguanide, the polybiguanide can be obtained in the form of any salt.

For preparing the metal salt complex with the polybiguanide, a method is described in U.S. Pat. No. 6,180,584, Example 2. Conveniently, an aqueous organic polar solvent solution of the polybiguanide salt is combined with the metal salt. As paradigmatic, one may consider the use of silver iodide. In the case of silver iodide desirably in the presence of a small amount of a water soluble iodide salt, generally from about 10 to 70 weight percent of the amount of the silver iodide. The product may be retained in solution or be isolated as described previously.

The following examples are offered by way of illustration and not by way of limitation.

### EXPERIMENTAL

### EXAMPLE 1

### Aqueous PHMB-AgI Solution

A. 20 g of Cosmocil CQ (Zeneca, Biocides, Wilmington, Del.) 4 g of silver iodide (AgI) 2 g of potassium iodide (KI) and 80 ml of N,N-dimethylformamide (DMF) were mixed together in a flask for 15 minutes. The volume of obtained solution (light yellow color) was adjusted with DMF to 100 ml. The resulting solution contained 10% (w/v) of solids. Prior to application, stock solution was 10-fold diluted with 1:1 (v/v) mixture of DMF and ethanol to a final solids content of 1% (w/v).

B. 20 g of Cosmocil CQ, 2.8 g of sodium dodecyl sulfate (SDS), 1.3 g of AgI, 0.4 g of KI and 25 ml of DMF, 20 ml N-methyl-2-pyrrolidone (NMP) and 20 ml of ethanol were mixed together in a flask for 30 minutes. The volume of obtained stock solution (yellow-brown color) was adjusted with ethanol to 100 ml. Prior to application, the stock solution was diluted with 70% (v/v) aqueous ethanol to a solids content of 0.5% (w/v).

C. 5 g PHMB 20% soln
0.027 g silver nitrate
0.057 g potassium iodide
1.786 g 30% PVP soln
0.5 g glycerin
5 g ethanol

5 g of a 20% aqueous PHMB solution, 0.057 g of potassium iodide, 0.027 g of silver nitrate, 1.786 g of 30% aqueous PVP K30 solution (BASF.) 0.50 g of glycerin and 5 g of ethanol were combined and allowed to react. The resulting solution was clear and colorless with water like viscosity. The weight ratios in weight percent of the components are as follows: PHMB, 1.00; ethanol, 5.00; PVP K30, 0.536; silver iodide, 0.057; potassium nitrate, 0.027; water, 92.88. The pH was adjusted to 7.0 with an approximate osmolality of 280.

D. Following the procedure described above, a suitable hydrogel formulation was prepared having the following weight percent ratios: PHMB, 0.067; ethanol, 0.336; PVP K30, 0.036; potassium iodide, 0.004; silver nitrate, 0.002; glycerin, 2.531; K4M (Dow Chemical Company) water, 95.00. The pH is 7.0 and the osmolality is 280.00

Following the procedure described above, a suitable mouthwash formulation was prepared having the follow weight percent ratios: PHMB, 0.067; ethanol, 30.168; PVP K30, 0.018; potassium iodide, 0.002; silver nitrate, 0.001; glycerin, 5.000; water, 64.778. The pH is 7.0 and the osmolality is 280.00

### Preparation of an Example API

Add 7.3 grams of a PVP solution detailed in Table 1 into an appropriate mixing vessel. Add to this solution the silver nitrate solution as detailed in the accompanying table and stir the mixture for 5 minutes. Dilute the mixture with the calculated amount of anhydrous ethanol and stir for another 5 minutes. To this mixture, the potassium iodide solution is slowly added and then the complete mixture is stirred for another 15 minutes. There should be no precipitate remaining at this point. If precipitate remains, continue mixing until all precipitate dissolves. The fractionated PHMB is then added to this solution followed by 30 minutes of stirring to dissolve any precipitated material. The solution is filtered through a 1 micron filter and is ready for use.

TABLE 1
PHMB-Agl based API formulation: biguanide unit/Agl molar ratio = 1.0:0.1

| | CQ | AgNO3 | KI | PVP | | |
|---|---|---|---|---|---|---|
| | (20% w/w) | (30% w/w) | (30% w/w) | (30% w/w) | EtOH | Total |
| density (g/ml) | 1.02 | 1.32 | 1.267 | 1.01 | 0.791 | n/a |
| weight parts | 1.000 | 0.077 | 0.529 | 0.766 | 0.775 | 3.147 |
| volume parts | 1.000 | 0.040 | 0.284 | 0.521 | 2.000 | 3.844 |
| volume per 1000 ml (ml) | 260 | 10 | 74 | 135 | 520 | 1000 |
| weight per 1000 g (g) | 318 | 25 | 168 | 243 | 246 | 1000 |

Formulation
composition, % wt.

| | PHMB.HCl | AgNO3 | KI | PVP | EtOH | water | total |
|---|---|---|---|---|---|---|---|
| component | 6.4 | 0.7 | 5.0 | 7.3 | 24.6 | 55.9 | 100.0 |
| content, | | | | | | | |
| % wt | | | | | | | |

| | |
|---|---|
| Cosmosil CQ | 20% w/w aqueous solution of |
| | PHMB.HCl |
| KI solution | 30% w/w aqueous solution |
| Silver nitrate solution | 30% w/w aqueous solution |
| Polyvinylpyrrolidone (PVP) | 30% w/w aqueous solution |
| solution (MW 30 kDa) | |

### Example Polymer Fractionation Procedure

Ultrafiltration fractionation of polymer PHMB is accomplished by using a holder sized appropriately for filter area needed, rotary lobe or peristaltic or any other pump that is able to deliver the flows and pressures needed, and appropriate pressure gauges and valves to control flow. System is connected together with stainless steel fittings and tubing or silastic tubing. For example, a Sartorius Hydrosart membrane with a 5 k molecular weight cut-off may be used. Hydrosart is a stabilized cellulose membrane that is hydrophilic and is stable over a broad pH range.

Cosmocil CQ (20% w/v) from vendor is obtained and diluted 1:2 with distilled water or other high quality purified water. After thorough mixing, the solution is recirculated through a UF system outfitted as above. Appropriate transmembrane pressures [TMPs] are used to maximize flux rates and to prevent the cartridge from fouling. After several minutes, the permeate valve is opened and the diafiltration process begins. Volume in the retentate vessel is maintained by adding dilution buffer, i.e. distilled water, as permeate is being collected. During processing, pressures are monitored and samples may be taken from the retentate as well as the permeate vessel. After an appropriate buffer exchange has been completed in order to remove the lower molecular weight material to an appropriate level, the bulk retentate can be concentrated to a more concentrated level via the UF system or directly transferred to a storage vessel for further processing. Further processing can include processing the material to solid form.

The UF system is then cleaned by recirculating DI through the system at a slightly higher TMP than processing conditions and also with an appropriate chemical agent (i.e. NaOH, organic solvent, high salt buffer, etc.). After removal of the chemical agent via deionized [DI] water recirculation, the system is then pressure tested to manufacturer's specifications and is stored until further use. Pressure testing may take place directly prior to use.

### EXAMPLE 2

### Treatment of Pig Wounds

A. Purpose

The purpose of this experiment was to test the prophylactic antibacterial efficacy of Neosil™ as an aqueous non-viscous solution and a gel.

Aqueous version

| | |
|---|---|
| PHMB | 1.000% |
| Ethanol | 5.000% |
| PVP K30 | 0.536% |
| potassium | 0.057% |
| iodide | |
| silver nitrate | 0.027% |
| Glycerin | 0.500% |
| Water | 92.880% |
| Total | 100.000% |
| pH | 7.00 |
| Osmolality | 280.00 |

Gel version

| | |
|---|---|
| PHMB | 1.000% |
| Ethanol | 5.000% |
| PVP K30 | 0.536% |
| potassium | 0.057% |
| iodide | |
| silver nitrate | 0.027% |
| Glycerin | 2.531% |
| K4M | 2.024% |
| Water | 88.826% |
| Total | 100.000% |
| pH | 7 |
| Osmolality | 280 |

The activity was compared to Bactroban (mupirocin), Polysporin, and vehicle controls. Pigs were chosen as the animal type to be used because of the similarity of pig skin to human skin, and because the porcine skin model is used in biomedical research in this area.

B. Pretreatment

Pigs were sedated and anesthetized following testing facility standard operating procedures. The pigs were then intubated endotracheally and maintained under a surgical plane of anesthesia with isoflurane 0.5-2.5% in room air. The back and flank hair was clipped and the skin was cleaned with alcohol. No Betadine® products were used.

C. Procedure

Standardized partial-thickness burns were created in approximately one inch circles with an aluminum cylinder at application intensity and duration that reliably produces first degree burns, i.e. 9 seconds 70° C. (Singer et al. Standardized burn model using a multiparametric histologic analysis of burn depth, Acad Emerg Med. January 2000; 7(1):1-6.)

In addition to the burn wounds, full-thickness skin defect (incisional) wounds approximately 1 cm in length were made on the backs of the animals and stapled. The burn and incisional lesions were in two columns—Left and Right paraspinal. The wounds were spaced approximately 1-2 inches from the midline in rows approximately 3 cm. apart.

A culture of Staphylococcus aureus ATCC6538 (standard FDA-approved strain for testing of biocides) was grown to a concentration of 10⁷ colony-forming units/ml. The bacteria was grown overnight in standard tryptic soy broth at 37° C.

Once all the burns/wounds were created on the pig, a cotton swab applicator was used to apply the bacteria to each wound. A sterile cotton swab applicator was immersed in the bacterial broth, and then rubbed on a wound for approximately 5-10 seconds.

Following application of bacteria, Formula Example 1(c) or control agents were applied to the wound. The introduction of bacteria was only performed once. The animals were allowed to recover from anesthesia and returned to normal housing for further recovery. No systemic antibiotics were used.

The day following the surgical procedure (day 2), treatment of the wounds with Formula Example 1(c) and positive controls were performed BID and continued until healing. Culturing of bacteria took place approximately every two days. On the days culturing took place, prior to each application of Formula Example 1(c) or control materials a sample of bacteria was collected from each wound by rubbing a cotton swab for approximately 5-10 seconds over the wound. The swab was then immersed in tryptic soy broth and bacteria cultured for colony counts.

In addition to determining whether Formula Example 1(c) reduces the bacterial load present in a skin infection (or prevents such an infection from taking place), wounds were visually inspected for signs of healing over the course of the experiment. The effect of the subject formulation, as compared to Neosporin/Polysporin controls, on the rate and quality of healing of the skin was visually assessed. Photographs were taken on each subsequent treatment/culturing days to track the progression of wound healing. The treatment/culturing procedure was continued for approximately 14 days. The gel and liquid formulations were found to be substantially equivalent in effectiveness.

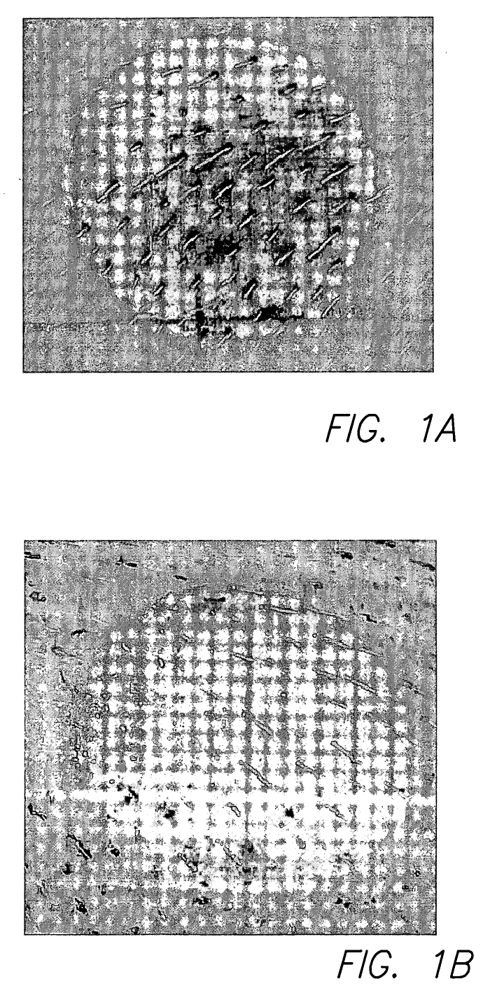

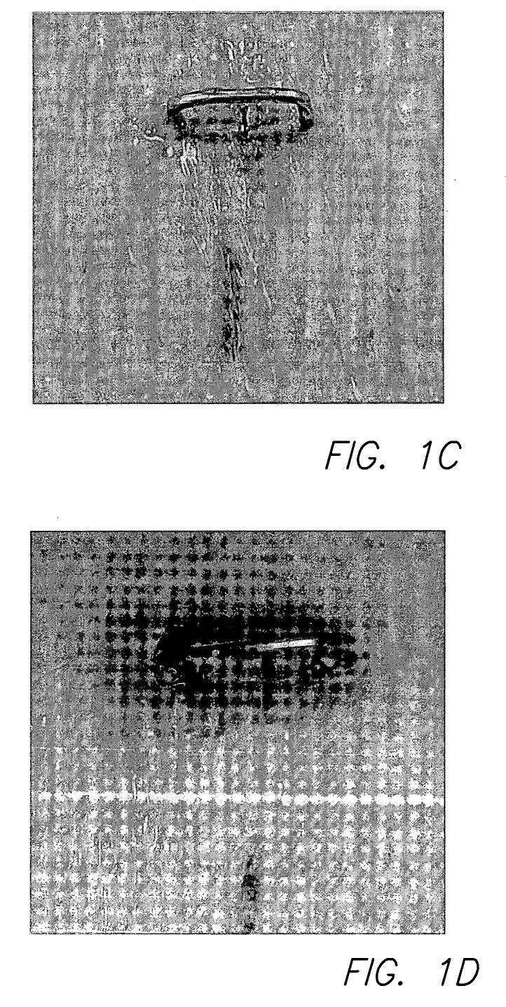

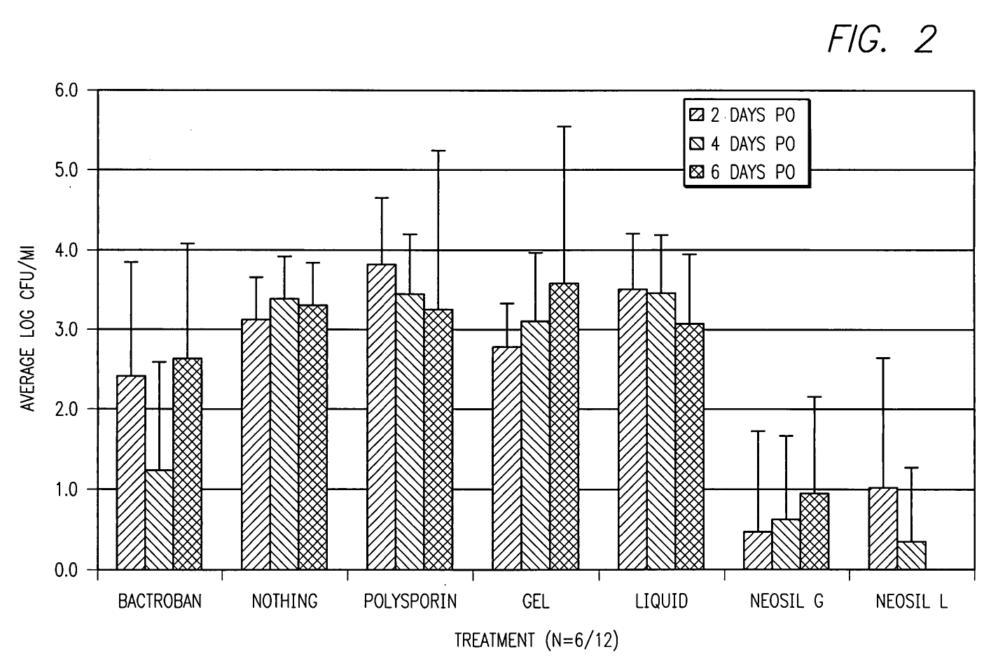

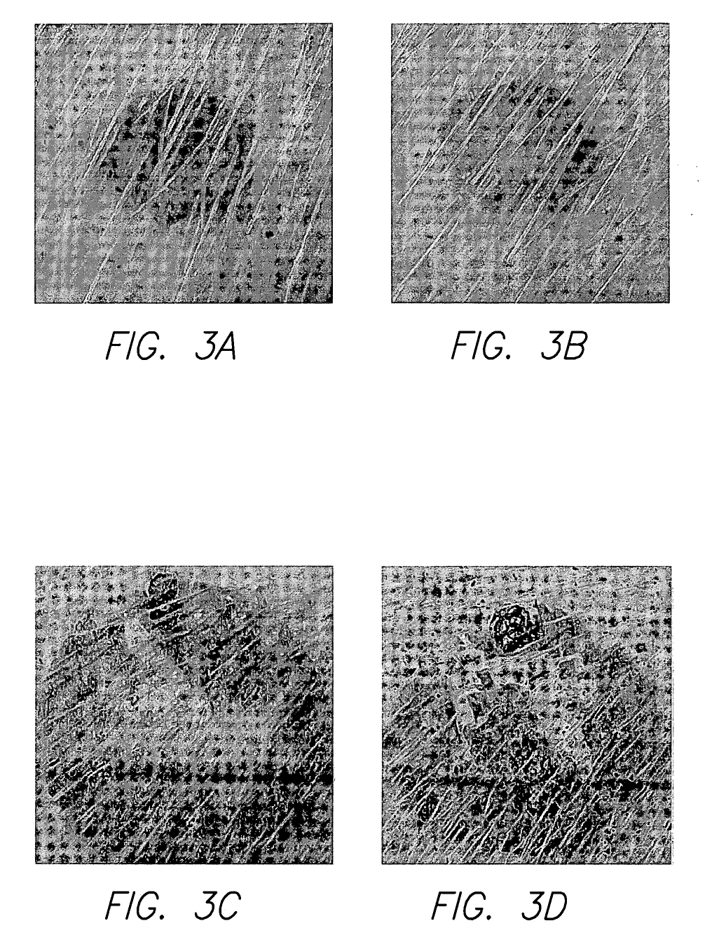

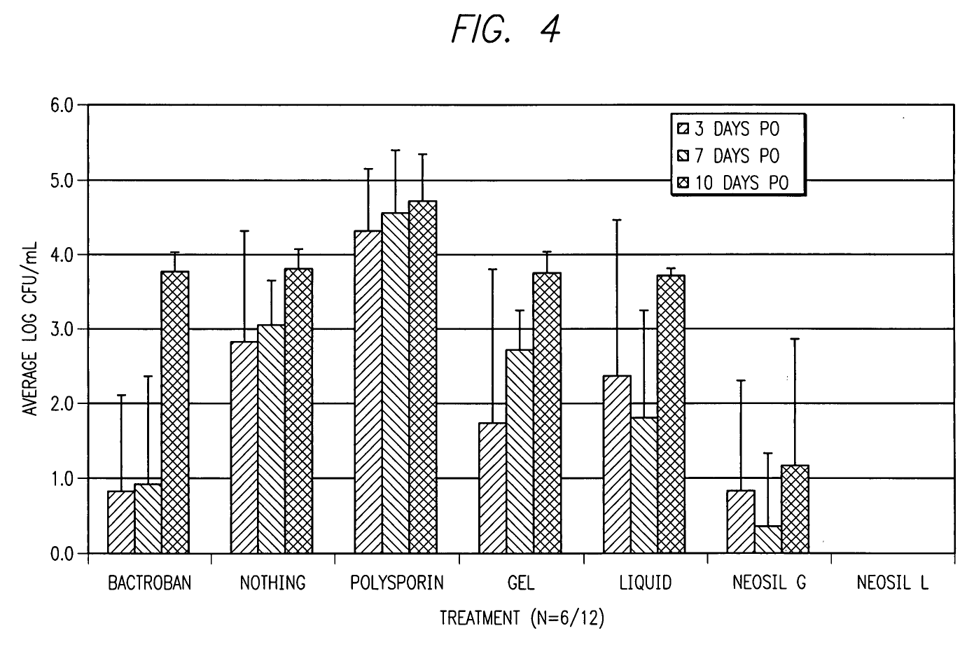

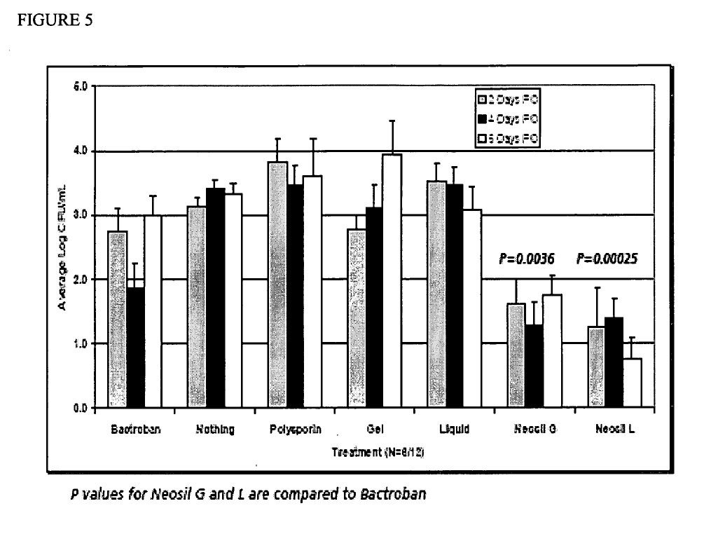

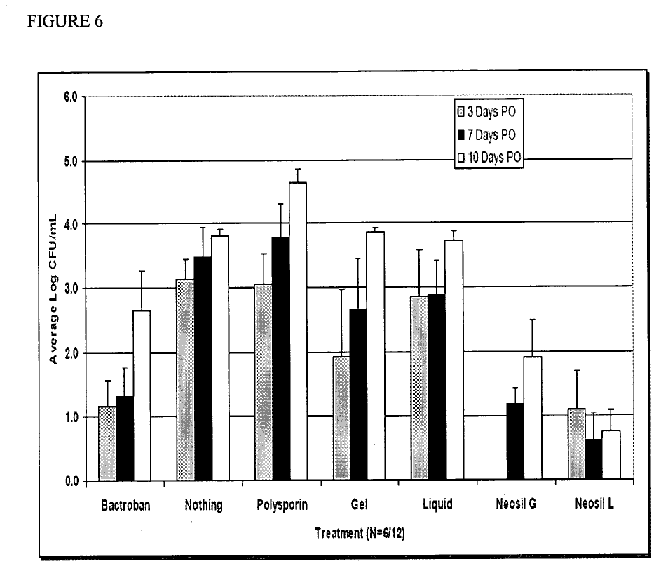

The results over the fourteen days are shown in FIGS. 1-6. It is evident from the results that the subject formulations are effective in protecting the wound from infection and do not interfere with the healing of the wound, where the wounds varied as to their nature. Each of the formulations comprising the subject compositions was effective in the treatment and was at least as good as and frequently better than commonly employed therapeutic agents.

### EXAMPLE 3

### Oral Antiseptic with Mice

A. Materials and Methods.

Mice.

Five-week-old female CD-1 mice were purchased from Charles River Laboratories. Mice were placed in cages in groups of five. To immunosuppress the mice and allow for the establishment of mucosal infection, 5-FU was given intravenously once every 7 days, starting on day-2. Antibiotics were given in the drinking water in autoclaved bottles to reduce potential confounding secondary bacterial infections. Gentamycin at 0.2 mg/ml, clindamycin at 1 mg/ml, vancomycin 1 mg/ml were added to sterile drinking water. Bottles and drinking water were changed every day. Imipenem is given at 5 mg/mouse (IP, QD). Antibiotics were begun on day-3.

Inoculum Preparation.

C. albicans #5 was transferred from storage at −80° C. and streaked for isolation on Sabouraud Dextrose Agar plates with chloramphenicol. The plates were incubated at 35° C. for 48 hours. The organisms were inoculated in sterile bottles each containing 100 ml of SAAMF broth and incubated for 48 hours at 35° C. on a gyratory shaker. C. albicans was harvested by transferring the broth culture to sterile 50 ml centrifuge tubes and centrifuged for 15 minutes at 2000 RPM. The cells were washed once with saline and then suspended in saline. The cells were counted using a hemacytometer. Inoculum dilutions were made in sterile water. The final inoculum was 2×10⁸ cells/ml of drinking sterile water plus antibiotics. The inoculum viability determined by plating serial dilutions on SDA plates with chloramphenicol was 1.85×10⁸ cells per ml. Plates were incubated overnight at 35° C. for verification count of the inoculum.

Infection of Mice.

In the morning before the preparation of the inoculum the drinking bottles were removed 8 hours prior to replacement with the inoculum suspension of C. albicans. The mice were allowed to drink from this suspension for 24 hours at which time the inoculum suspension was removed and replaced with drinking water containing antibiotics (day 0).

Beginning on day 4 postinfection, mice were untreated or treated with either, Surfacine D™ diluent (undiluted), 3% Surfacine D, PEG dilutent or 1% clotrimazole.

| | |
|---|---|
| PHMB | 3.000% |
| PVP K30 | 1.607% |
| potassium iodide | 0.171% |
| silver nitrate | 0.080% |
| EtOH | 30.000% |
| glycerin | 5.000% |
| water | 60.141% |
| total | 100.000% |
| pH | 7 |
| osmolality | 280 |

PEG 400, 1% clotrimazole in PEG400, or given no treatment. Treatments were done for 10 consecutive days and were given twice daily. Treatments were done by dipping a sterile calcium alginate swab into the solution and then swabbing the oral cavity of the mouse to ensure coverage with the solution. It was not necessary to anesthetize the animals to perform the treatments. Treatment ended on day 13 postinfection.

On day 15 postinfection all surviving mice were euthanatized using CO₂ gas. The tongue of each mouse was swabbed with sterile calcium alginate swab and the swab placed in 0.4 ml of 1×PBS. The swab in PBS was vigorously mixed with a vortex mixer to dissolve the alginate and release the organisms into suspension, and two 10-fold dilutions were made and plated in duplicate on SDA without chloramphenicol.

Survival was analyzed using a log rank test and comparative CFU between groups was analyzed using a Mann-Whitney U test. A log₁₀ value of 6.5 was assigned as CFU for data points missing due to the death of the animal. This value is arbitrarily set to be higher than any burden recovered from surviving mice and assures that death is considered as a worse outcome than is survival regardless of burden.

### SUMMARY

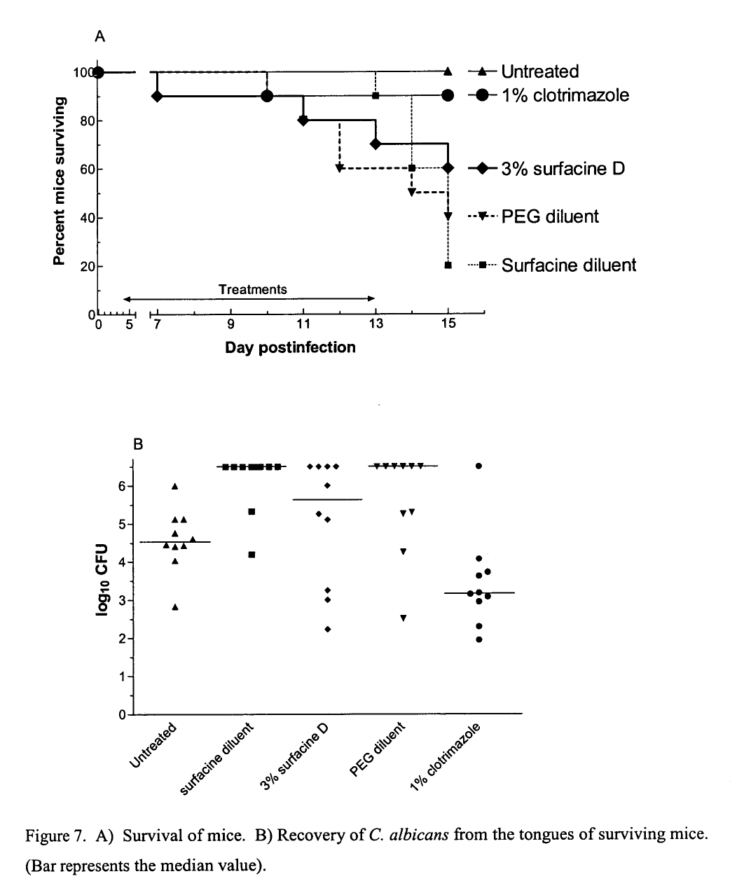

A murine model of mucosal candidosis of the oral cavity was established in immunosuppressed mice. The results for survival and of CFU recovered from the mice in the comparative treatment regimens are shown in FIGS. 7A and B, respectively. The model performed as expected with regard to the group given no antifungal therapy (untreated controls). None of these animals died during the course of the experiment and the median CFU recovered from the tongue were about log₁₀ 4.5, which is comparable to previous data. The positive control group, 1% clotrimazole in PEG400, had one death. This group showed about a 30-fold reduction in CFU in comparison with the untreated controls (P=0.014). The PEG400 control group had 60% deaths and no apparent change in CFU. The Surfacine D-diluent group and the 3% Surfacine D-treated groups also had deaths occur. For the Surfacine-diluent group 80% of the mice died, whereas 40% died in the Surfacine D group. It should be noted that the first death in the Surfacine-diluent group occurred on day 13 of infection, whereas the first death in the 3% Surfacine D group occurred on day 7 postinfection. The large numbers of deaths in these groups make the CFU comparisons difficult, but as shown in FIG. 7B the CFU range for the Surfacine D-treated encompassed the range for those animals given no treatment. Clotrimazole was the most effective of the treatments and no animals in any group were found to be free of detectable C. albicans on the mucosal surfaces.

We could not attribute the deaths in the study to secondary bacterial infection, as lower numbers or no bacteria were found on plating of organ homogenates from randomly chosen dead animals nor were CFU of Candida albicans large enough to be considered as the cause of death. What may have been an important contributing factor to these results was the stress of the handling for the various treatments (two times a day for topical treatment and once daily for antibiotic administration i.p.). However, the clotrimazole-treated mice were also handled the same number of times each day, which would be suggestive that death was not due solely to handling stress. Behavioral observations made during the treatments were indicative that the animals receiving the 3% Surfacine D and its diluent aggressively resisted the swabbing treatment as if the taste was extremely unpalatable. Additionally, some mice in these groups developed a noticeable diarrhea approximately half way through the experiment; this was greater than the softened stools observed from all mice because of the broad-spectrum antibiotic treatments. The overall appearance of the animals was also poorer than that of the untreated or the clotrimazole-treated. Whether the deaths, behavioral changes and resistance to treatment is indicative of some aspect of toxicity by the diluent or the Surfacine D remains to be determined. It is also possible that the deaths were due to progressive infection arising from the expected translocation of the organisms from the gut to cause systemic disease. Somewhat similar were the reactions of the mice to PEG alone in that they were less willing to be treated and appeared to have difficulty in opening their mouths, whereas the clotrimazole-treated mice did not exhibit these behaviors or symptoms.

The gross pathological appearance of the tongues at necropsy was areas of white patchiness on the mucosal surfaces of the PEG- and untreated animals. All clotrimazole-treated animals had normal mucosal surface appearance. For the Surfacine diluent, one appeared normal and one had areas of patchiness. Five of the 6 Surfacine D-treated had normal mucosal appearance and 1 had slight patchiness (i.e., 1 small distinct area). Thus, with respect to the gross observations, the Surfacine D did appear to be effective. Assessment of the evolution of the disease development or resolution during therapy could not be made, because the tongue would need to be extended for satisfactory examination, whereas the mice are battling the treatment procedure.

TABLE 2
Statistical analyses of survival by log rank test.

| | Significantly prolonged survival versus (P value =) | | | | |
|---|---|---|---|---|---|
| | | | 3% | | 1% |
| | Un- | Surfacine- | Surfacine | PEG- | clotri- |
| | treated | diluent | D | diluent | mazole |
| Untreated | — | | | | |
| Surfacine- | 0.0004 | — | | | 0.005 |
| diluent | | | | | |
| 3% Surfacine D | 0.029 | | — | | |
| PEG-diluent | 0.0045 | 0.004 | | — | 0.028 |
| 1% clotrimazole | | | | | — |

Comparisons not shown were not significant at the 0.05 level.

TABLE 3
Statistical analyses by Mann-Whitney U test of
comparative CPU recovered from the tongue.

| | Significantly reduced CPU versus (P value =) | | | | |
|---|---|---|---|---|---|
| | | | 3% | | 1% |
| | Un- | Surfacine- | Surfacine | PEG- | clotri- |
| | treated | diluent | D | diluent | mazole |
| Untreated | — | | | | 0.014 |
| Surfacine- | 0.001 | — | | | 0.0003 |
| diluent | | | | | |
| 3% Surfacine D | | | — | | 0.06 |
| PEG-diluent | 0.023 | | | — | 0.005 |
| 1% clotrimazole | | | | | — |

Comparisons not shown were not significant at the 0.05 level.

### EXAMPLE 4

### Oral Antiseptic with Dogs

### Evaluation of Experimental Test Solutions on Oral Malodor in Dogs

A. Purpose

The purpose of this study was to evaluate the effect of an experimental test solution on oral malodor in the dog. The two test groups were comprised of an experimental rinse and a placebo rinse.

B. Test Substances

Research Compliance was responsible for storage requirements, expiration dates and any other applicable requirements. To complete this study approximately 450 ml of each test rinse was required.

C. Justification for Animal Use

This program was designed to evaluate a regimen which may have potential for improving the oral health of dogs by reducing oral malodor. No suitable in vitro model exists for studies of this nature. Therefore, the dog was the appropriate model. This study was designed as a screening study using a longitudinal study design. The number of animals used was limited to the current population of the OHRI colony (24 mixed-sex dogs).

D. IACUC Approval

The protocol was reviewed and approved by the Institutional Animal Care and Use Committee prior to initiation of study.

E. Test Design

Experimental procedures were conducted using GLP guidelines. The dogs were fed a nutritionally complete commercially available dry dog food daily. The test solution was administered mid morning daily.

TABLE 4

| Group | N | Test Rinse |
|---|---|---|
| A | 12 | 1370 - A Control Rinse |
| B | 12 | 1370 - B Test rinse |

F. Animals

1. Type of Animals

Adult mixed-sex beagle dogs. The age of the dogs ranged from three (3) to eleven (11) years of age.

2. Number of Animals

There were a total of 24 dogs.

3. Source of Animals

Original source of animals were obtained in accordance with USDA regulations. All dogs are maintained as the OHRI colony.

4. Identification

All of the dogs have been given an identifying number, unique to that animal, as an ear tattoo. The number was also marked on a tag attached to the dog's cage.

5. Housing

All dogs were housed in individual cages in an AAALAC-accredited facility. Room temperature was maintained at 72° F. (±6° F.) with 10-15 air changes per hour and a 12-hour light cycle.

6. Husbandry and Health Care

All animal husbandry procedures were provided in accordance with the testing facility standard operating procedures. The health of the animals was assured with routine CBC and chemistry profiles. These were obtained upon the receipt of the animals and yearly thereafter. The animals were observed daily by a staff member and weekly by the attending veterinarian for any signs of health problems.

G. Procedures

1. Stratification

The animals were evenly stratified by block design into 2 groups of 12 dogs. The animals were balanced on the basis of baseline oral malodor scores prior to study initiation of the experimental phase.

2. Feeding

The animals were fed approximately at the same time daily. The amount of diet fed was calculated on an individual animal basis, (18 g/Kg). This amount was adjusted as needed to maintain a stable body weight. Any remaining food was weighed and recorded prior to the next daily feeding.

3. Watering

The dogs were given tap water ad libitum. Fresh water was given twice daily. The water was withheld for approximately 1.5 hours following administration of the test rinses.

4. Body Weight

The dogs were weighed one day prior to study initiation and at study completion.

5. Test Solutions

The test rinses were 2 coded products supplied by the Sponsor. To perform this pilot study, 450 ml. of each rinse was required. The sponsor was responsible for the necessary evaluation related to the composition, purity, strength, stability, storage requirements, expiration dates and any other applicable requirements.

6. Treatment Application

The treatment phase was initiated following baseline stratification. The experimental rinses were administered at approximately the same time daily (at 22-23 hour intervals) for three (3) consecutive days. Each treatment group had a coded beaker, which was designated for that treatment only. Each test group had a color-coded tag attached to the animal's cage to correspond with the coded test group. All drinking water was removed from the animal cages prior to treatment and not returned for at least 90 minutes post treatment. The test solutions were applied to all of the maxillary and mandibular teeth in their assigned treatment group. A 10 cc syringe was used to apply the solution. Specifically, 2.5 cc for each quadrant was applied. The test rinse (within the appropriate group) was evenly dispersed to each hemijaw over the teeth to be evaluated and allowed to pool in the mandibular region. Special care was taken to prevent the animal from swallowing excessive amounts of the solution.

7. Examinations

a. Conduct

Oral Malodor was evaluated via human perception (Appendix B) as well as using instrumentation (Appendix A). Three (3) oral malodor readings were taken. The readings were taken utilizing a volatile sulfur meter (Halimeter, Interscan Corporation®). The oral malodor (ppb-VSC) was evaluated at:
Day-1 Baseline readings
Day-3: 90 minutes after administration of the test rinse. 8 hours post administration of the test rinse.
Day-4 23-hours after administration of the test rinse.

The dogs were examined by block in a random sequence to avoid systematic bias. The animals were taken to the examination area by a certified laboratory animal technician. The animals were examined for oral malodor (Appendix A). Examiner observations were recorded on prepared exam forms by the recorder who was not directly involved in the examinations.

b. Examination Sequence-Test Period
Human Sniff Assessments
Halimeter

C. Oral Assessment Methods

| | |
|---|---|
| Oral malodor (Halimeter) | Appendix A |
| Human sniff assessments | Appendix B |

H. Experimental Duration of Study

A longitudinal study design was used. This period was comprised of a 3-day treatment test phase plus a 23-hour post-test evaluation. The total duration of each test period was 4 days. Following study completion, the dogs were returned to the Bioresearch Facility colony.

I. Data Processing

Using the SAS statistical package the data were analyzed using ANOVA models, which included effects for baseline score and treatment group. The specific type of data calculated and analyzed were: J.

J. Oral Malodor

Sniff & VSCppb (Mean±S.E.M.)
Baseline
1.5 hrs post 3^{rd} treatment
8 hrs post 3^{rd} treatment
23 hour post 3^{rd} treatment

Body Weight
Initial Weight
Final Weight
Weight Change
   K. Statistical Methods

Comparisons between the two groups for differences in initial weight, weight gain, and baseline malodor were performed using two-sample t-tests. Comparisons to test for weight change within a group were performed using paired t-tests. Comparisons between groups for differences in the change in oral malodor halimeter measurements were performed using analysis of covariance. The model included baseline malodor as a covariate, hours, group, and the hours-by-group interaction. A random dog effect was included to correlate the multiple measurements within a dog. The Sidak method was used to control the overall significance level of the pairwise tests: adjusted p-value=1−[1−unadjusted p-value]^{#tests}. Comparisons within group for significance of changes from baseline were also tested within the analysis of covariance model. Mantel-Haenszel chi-square tests for ordered categorical responses were used to compare the groups for differences in baseline oral malodor human sniff assessments. Mantel-Haenszel tests also were used to compare the groups for differences in the change in sniff assessments. Comparisons to test for changes in sniff assessments within a group were performed using Wilcoxon signed rank tests.

L. Record Maintenance

All records (protocols, amendments, stratification, data sheets, and final reports) will be maintained in a book designated for this study as part of the OHRI Laboratory Archives.

M. Results & Conclusions

The results observed in this study are discussed with each of the following tables. As noted, the experimental solution used to treat the Group B animals significantly reduced oral malodor as assessed by the instrumental measurement of sulfur-containing compounds and a human sniff test as compared to the Group A animals treated with the placebo solution. The magnitude of the reduction in malodor was greatest at 8 hours following the treatments.

Results

Dogs from both groups lost weight during the study (p=0.0379 for Group A; p=0.0331 for Group B). However there was no difference between groups for initial weight (p=0.59) or weight change (p=0.87). The groups also did not have significantly different baseline oral malodo halimeter measurements (p=0.98) or baseline oral malodor human sniff assessments (p=0.80).

TABLE 5
Body Weight

| | | | | Weight Change | | | |
|---|---|---|---|---|---|---|---|
| | | | | Initial Weight | | Final Weight | Weight Change |
| Group | N | Snack Treat | | Kg | | Kg | Kg |
| A | 12 | Control Rinse * | ** | 14.03 ± 0.64 *] | ** | 13.89 ± 0.65 ] | −0.14 ± 0.06 ] |
| B | 12 | Test Rinse | | 13.55 ± 0.59 | | 13.43 ± 0.60 | −0.12 ± 0.05 |

Oral malodor halimeter measurements decreased significantly within both groups at each follow-up examination (Group A: p=0.0145 at 1.5 hours, p=0.0237 at 8 hours, p=0.0012 at 23 hours; Group B: p<0.0001 at 1.5, 8, and 23 hours). The overall test for a significant difference between groups for change in oral malodor halimeter measurements was significant (p<0.0001), with a significantly larder decrease for Group B. For the individual follow-up examinations, Group B had a significantly larger decrease for 1.5 hours (p=0.005 1) and 8 hours (p<0.0001) and had a marginally significantly larger decrease for 23 hours (p=0.0899).

TABLE 6
Oral Malodor

| Group | Hours | N | Mean Score | Standard Error |
|---|---|---|---|---|
| | 0 | 12 | 213.92 | 33.36 |
| Control Rinse | 1.5 | 12 | 184.61 | 30.68 |
| Group A | 8 | 12 | 186.92 | 30.32 |
| | 23 | 12 | 174.14 | 30.49 |
| | 0 | 12 | 212.67 | 28.73 |
| Test Rinse | 1.5 | 12 | 129.61 | 13.36 |
| Group B | 8 | 12 | 99.53 | 7.85 |
| | 23 | 12 | 137.28 | 16.03 |

For changes within a group, Group B improved significantly from 1.5 hours to 8 hours (p=0.0206) but reversed from 8 hours to 23 hours (p=0.0027) so that 1.5 hours and 23 hours were not significantly different (p=0.85). However Group A did not change significantly between follow-up examinations (p=0.99 for changes between 1.5 hours and 8 hours, p=0.70 for changes between 1.5 hours and 23 hours, and p=0.55 for changes between 8 hours and 23 hours).

TABLE 7
Oral Malodor Change

| Group | Hours | N | Mean Score | Standard Error |
|---|---|---|---|---|
| Control | 1.5 | 12 | −29.31 | 11.27 |
| Group A | 8 | 12 | −27.00 | 9.11 |
| | 23 | 12 | −39.78 | 8.04 |
| Test Rinse | 1.5 | 12 | −83.06 | 16.22 |
| Group B | 8 | 12 | −113.14 | 24.42 |
| | 23 | 12 | −75.39 | 17.27 |

Oral malodor human sniff assessments within Group A did not change significantly from baseline to 1.5 hours (p=0.63), baseline to 8 hours (p=0.50), or baseline to 23 hours (p=1.00). Sniff assessments within Group B also did not change significantly from baseline to 1.5 hours (p=0.63) or baseline to 23 hours (p=0.22), but there was a significant reduction in scores from baseline to 8 hours (p=0.0156).

TABLE 8
Human Sniff Assessment

| | |
|---|---|
| Group A | Group B |

| Hours | Score * | # | % | # | % |
|---|---|---|---|---|---|
| | 1 | 3 | 25.00 | 5 | 41.67 |
| 0 | 2 | 6 | 50.00 | 3 | 25.00 |
| | 3 | 3 | 25.00 | 4 | 33.33 |
| | 1 | 5 | 41.67 | 4 | 33.33 |
| 1.5 | 2 | 4 | 33.33 | 7 | 58.33 |
| | 3 | 3 | 25.00 | 1 | 8.33 |
| | 1 | 3 | 25.00 | 10 | 83.33 |
| 8 | 2 | 4 | 33.33 | 2 | 16.67 |
| | 3 | 5 | 41.67 | 0 | 0.00 |
| | 1 | 4 | 33.33 | 6 | 50.00 |
| 23 | 2 | 3 | 25.00 | 5 | 41.67 |
| | 3 | 5 | 41.67 | 1 | 8.33 |

* 0 = No perceivable odor 1 = Mild, odor @ 6″ 2 = Moderate, strong odor 6″ to 12″ 3 = Severe = Intense odor greater than 12″

The overall test for a significant difference between groups for change in oral malodor human sniff assessments was significant (p=0.0036), with significantly more decrease for Group B. For the individual follow-up examinations, there was not a significant difference between groups for 1.5 hours (p=1.00), but Group B had significantly more decrease for 8 hours (p=0.0028) and had marginally significantly more decrease for 23 hours (p=0.0956).

TABLE 9
Human Sniff Change

| | |
|---|---|
| Group A | Group B |

| Hours | Score * | # | % | # | % |
|---|---|---|---|---|---|
| | −1 | 3 | 25.00 | 3 | 25.00 |
| 1.5 | 0 | 8 | 66.67 | 8 | 66.67 |
| | 1 | 1 | 8.33 | 1 | 8.33 |
| | −2 | 0 | 0.00 | 2 | 16.67 |
| 8 | −1 | 0 | 0.00 | 5 | 41.67 |
| | 0 | 10 | 83.33 | 5 | 41.67 |
| | 1 | 2 | 16.67 | 0 | 0.00 |
| | −1 | 1 | 8.33 | 5 | 41.67 |
| 23 | 0 | 9 | 75.00 | 6 | 50.00 |
| | 1 | 2 | 16.67 | 1 | 8.33 |

* 0 = No perceivable odor 1 = Mild, odor @ 6″ 2 = Moderate, strong odor 6″ to 12″ 3 = Severe = Intense odor greater than 12″

### Appendix A

### Oral Malodor Assessment

Scoring Method

A Halimeter will be used to measure Volatile Sulfur Compounds (VSC). The meter will be turned on for at least 20 minutes prior to use. The sampling tube will be placed parallel for the buccal Maxillary P₄. Cheek mucosa will be kept away from the end of the sampling tube and the animal's mouth closed. The highest reading after a stabilization period (10-15 seconds) will be recorded. Right, left and lingual anterior areas will be sampled.

Calculations

The score for the animal is the mean of these readings.

### Appendix B

### Oral Malodor-Human Assessment

Scoring method

0—No perceivable oral odor

1—Mild—odor not detectable 6″ from open lip

2—Moderate—odor strong near mouth and is detectable 6-12″ from dogs'mouth

3—Severe—odor intense near mouth and is detectable >12″ from dogs' mouth

Method

The animal's lip (right or left side) will be retracted. The examiner will then sniff the dogs breath beginning at the farthest measurement point >12″. The score for each animal will be recorded.

It is evident from the above results that the subject compositions can provide long term protection in environments where the area of interest is in contact with or encompassed by living tissue, where added compositions are subject to dilution, removal, degradation and modification. The subject compositions result in the substantial reduction of bacterial population in a variety of environments, while retaining the protection over extended periods of time. In each case, adverse effects are limited or absent, and the compositions are well tolerated.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for treating mucosal or open wound tissue susceptible to infection by cellular microorganisms to inhibit proliferation of such cellular microorganisms, said method comprising:
applying to said tissue in an amount effective to inhibit such cellular microorganisms a composition effective for such inhibition, said composition comprising as the active ingredient an antimicrobial polycationic polymer complexed with from 0 to 20 weight % of said complex of an antimicrobial metal material.

2. A method according to claim 1, wherein said polycationic polymer is a polybiguanide having at least 4 biguanide units.

3. A method according to claim 2, wherein said polycationic polymer comprises anions that reduce the hydrophilicity of said polycationic polymer.

4. A method according to claim 1, wherein said tissue is mucosal tissue or dermal tissue.

5. A method according to claim 1, wherein said applying comprises the use of a dispersion, spray, cream, lotion, foam, ointment or gel.

6. A method for treating mucosal or open wound tissue and susceptible to infection by cellular microorganisms to inhibit proliferation of such cellular microorganisms, said method comprising:,
applying to said tissue in an amount effective to inhibit such cellular microorganisms a composition effective for such inhibition, said composition comprising as the active ingredient an antimicrobial polybiguanide polymer complexed with at least 1 weight % of said complex of an antimicrobial water insoluble silver or silver salt.

7. A method according to claim 6 wherein said silver is silver nanoparticles.

8. A method according to claim 6, wherein said silver salt is silver iodide or bromide.

9. A method according to claim 8, wherein said polybiguanide polymer comprises at least 4 biguanide groups.

10. A method according to claim 6, wherein said tissue is an open wound.

11. A method according to claim 6, wherein said treating is for acne, impetigo, burns, fungal infections or dermatophytes.

12. A method according to claim 6, wherein said treating is for vaginal infection.

13. A method according to claim 6, wherein said method employs topical treatment.

14. A method for treating mucosal or open wound tissue and susceptible to infection by cellular microorganisms to inhibit proliferation of such cellular microorganisms, said method comprising:
applying to said tissue in an amount effective to inhibit such cellular microorganisms a composition effective for such inhibition, said composition comprising as the active ingredient an antimicrobial polybiguanide polymer having at least 4 biguanide groups complexed with at least 1 weight % of said complex of antimicrobial water insoluble silver iodide.

15. A method according to claim 14, wherein said open wound tissue is a burn.

16. A method according to claim 14, wherein said open wound tissue results from removal of the stratum corneum.

17. A method according to claim 14, wherein said mucosal tissue is in the mouth.

18. A method according to claim 14, wherein said applying is as an aqueous dispersion.

19. A pharmaceutical composition comprising polybiguanides of which at least 90 weight % have a molecular weight in the range of 1.5 kamu to 20 kamu and from 0 to 20% of silver or salt.
